(19) United States
(12) Patent Application Publication
(10) Pub. No.: US 2019/0091064 A1
(43) Pub. Date: Mar. 28, 2019

(54) MEDICAL CATHETER FOR HYPOTHERMIC TREATMENT

(71) Applicant: ACANDIS GMBH & CO.KG, Pfinztal (DE)

(72) Inventors: Giorgio CATTANEO, Karlsruhe (DE); Michael BÜCHERT, Bretten (DE); Julia WOLFERTZ, Karlsruhe (DE)

(73) Assignee: Acandis GmbH, Pforzheim (DE)

(21) Appl. No.: 16/094,205

(22) PCT Filed: Apr. 18, 2017
(86) PCT No.: PCT/EP2017/059203

§ 371 Date: Oct. 16, 2018

(30) Foreign Application Priority Data
Apr. 18, 2016 (DE) 10 2016 107 107.2

(51) Int. Cl. A61F 7/12; A61M 25/00

(52) U.S. Cl. CPC A61F 7/12; A61M 25/0026; A61F 2007/0086; A61F 7/123; A61M 25/005

(57) ABSTRACT

A medical catheter for hypothermic treatment has a catheter tube, which has at least two inner lumina. At least one inner lumen forms a temperature control duct that is suitable for in particular provided for, conveying a cooling liquid. A reinforcement is associated with at least one inner lumen.

### BACKGROUND OF THE INVENTION

### 1. Field of the Invention

The invention relates to a medical catheter. In particular, the invention relates to a medical catheter having for hypothermia treatment with a catheter tubing having at least two inner lumens, wherein at least one inner lumen forms a temperature control duct suitable for conveying a cooling liquid.

### 2. Discussion of the Related Art

Such a catheter is known from DE 10 2013 104 948 A1, for example.

The known catheter has a total of three inner lumens, wherein two of the inner lumens form temperature control ducts. The temperature control ducts are connected to a heat exchanger element on the distal end of the catheter, so that a temperature control circuit can be formed.

The catheter is preferably used for local cooling of tissue areas. To do so, the catheter is inserted into a blood vessel and has a coolant flowing through it. Therefore, the blood flowing around the catheter is cooled. This reduces the metabolic activity in downstream tissue areas, which is advantageous in treating strokes and myocardial infarctions or in the context of reanimation, in particular in the post-reanimation phase.

Hypothermia treatment of strokes is usually administered by inserting a catheter into the femoral artery. The catheter must then be advanced into the carotid artery by way of the aortic arch. There is a marked curvature in the catheter in the transitional area between the aortic arch and the carotid artery. With known catheters, this often results in deformation of the inner lumen of the catheter. In particular, the inner lumen assumes an oval cross-sectional contour. In the case of inner lumens used to guide a cooling liquid, this results in a pressure drop in the transitional area between the aortic arch and the carotid artery, which has a negative influence on cooling performance.

Medical instruments can be introduced into a patient's circulatory system through the additional inner lumen of the catheter, which is designed as a through-lumen. The marked curvature in the transitional area between the aortic arch and the carotid artery often results in deformation of the through-lumen. This makes it difficult to insert medical instruments through the through-lumen. In the extreme case, a kink in the catheter in the narrow transitional area between the aortic arch and the carotid artery may impede or prevent further navigation of the catheter to the target position. Furthermore, a complete kink in the catheter can stop the flow of coolant and thus cause the cooling function to be suspended.

### SUMMARY OF THE INVENTION

The invention is based on the object of providing a medical catheter for hypothermia treatment, which will provide strong cooling power and/or good navigability for medical instruments after being passed through vessels with tight curvatures.

The aforementioned object is achieved in particular by a medical catheter for hypothermia treatment with a catheter tubing having at least two inner lumens, at least one inner lumen forming a temperature control duct, which is provided in particular for conveying a cooling fluid. A reinforcement is provided for at least one inner lumen, in particular for the temperature control duct.

The reinforcement may be a separate component from the catheter tubing and/or may form a separate component of the catheter. The reinforcement may be a reinforcing element to this extent. The reinforcement and/or the reinforcing element may be fixedly connected to the catheter. However, the reinforcement has other mechanical properties than those of the catheter tubing.

The reinforcement is preferably formed by an additional geometric adaptation of the inner lumen and/or by an additional material, in particular a material that is different from the material of the catheter tubing. The reinforcement is preferably designed so that the bending behavior of the catheter tubing is improved. In particular the flexural stability of the catheter tubing is improved by the reinforcement. In general, the reinforcement alters the mechanical properties of the catheter tubing.

The reinforcement achieves the result that the catheter can also be moved well in a blood vessel even through tight curves. The navigability of the catheter is maintained in this way. Moreover, the reinforcement ensures that the inner lumen is not deformed. This prevents a negative effect on the cooling power in the case of inner lumens carrying the cooling fluid. In the case of an inner lumen designed as a through-duct to guide medical instruments to the site of treatment, the navigability of the medical instruments is maintained even when the catheter is being guided through a tight curvature.

It is pointed out that the catheter according to the invention is in general suitable not only for cooling a human or animal body but also for heating or maintaining the body temperature. The temperature control effect of the catheter need not affect the entire body but instead can also be limited locally to individual tissue areas and/or organs.

It is possible to provide in particular that one of the inner lumens forms a through-duct which is separate from the temperature control duct, wherein a reinforcement is provided at least for the through-duct and/or the temperature control duct. In concrete terms, only the through-duct may have a reinforcement.

In a preferred embodiment, it is provided that the reinforcement has at least one material different from the material of the catheter tubing. In concrete terms, the reinforcement may line the inner lumen, in particular the temperature control duct, and may be made of a material different from the material of the catheter tubing.

The material of the reinforcement may have a greater flexural strength than the material of the catheter tubing. In particular the reinforcement may be formed by an inner tubing or an inner coating of the inner lumen. In other words, the inside circumference of the inner lumen may be lined by the reinforcement. The reinforcement may comprise or consist of plastic or metal as the material. A material mixture of plastic and/or metal may also be used for the reinforcement. At any rate, it is provided that the reinforcement supports the inner lumen to thereby prevent deformation of the inner lumen.

In this context, it is pointed out that within the scope of the present patent application, it is explicitly ruled out that the reinforcement may be formed by simply increasing the wall thickness of the catheter tubing. Instead, the reinforcement forms a separate element, formed either by a geometric design of the inner lumen or by an additional material.

Furthermore, the material of the reinforcement may also be designed to reduce friction. This facilitates the navigation of medical instruments through the inner lumen.

The reinforcement preferably has thermally insulating properties. Such a thermally insulating reinforcement is provided in particular for lining temperature control ducts of a catheter tubing in order to reduce thermal losses.

In another preferred embodiment of the invention, it is provided that the reinforcement has structuring, in particular a longitudinal grooved contour. The structuring may be formed in the material of the catheter tubing and/or in the material of the reinforcement. In other words, it may be provided, on the one hand, that the reinforcement is formed by structuring of the inner lumen of the catheter tubing. In particular, the inner lumen may have longitudinal grooves which result in stiffening of the inner lumen. On the other hand, the reinforcement may also be formed by an additional layer of material in the inner lumen. The additional layer of material, which is preferably a material different from the material of the catheter tubing, may additionally have structuring, for example, a longitudinal grooved profile.

The use of longitudinal grooves as a reinforcing element increases not only the flexural stability and/or kink stability but also results in a reduction in friction between the inner lumen and a medical instrument. This is the result of the fact that the contact area between a medical instrument and the inner lumen is reduced due to the longitudinal grooves.

The reinforcement may be constructed in particular of at least two, in particular more than two materials in multiple layers. The number of layers preferably corresponds to the number of materials used for the reinforcement. For example, an inner layer of the reinforcement may be made of PTFE for other friction-reducing materials, FEP or HDPE. A middle layer of the multilayer reinforcement may comprise a metal. An outer material of the reinforcement, which comes in direct contact with an inner circumferential surface of the inner lumen, may comprise a thermoplastic elastomer.

In a particularly preferred embodiment of the catheter according to the invention, the reinforcement has a metallic mesh or a metallic winding. The metallic mesh or metallic winding is preferably formed as the central layer of a multilayer reinforcement. The mesh or the winding may comprise at least one round wire and/or one flat wire each. If the metallic material is to be omitted, it is also possible to provide for the mesh or the winding to be formed by polymer fibers. The winding preferably extends in the shape of a spiral, i.e., a helix, around the longitudinal axis of the inner lumen.

It is preferably provided in general that the reinforcement extends over the entire inner circumference of the inner lumen. In concrete terms, the inner lumen may be lined completely by the reinforcement.

Furthermore, the reinforcement may extend over the total length of the inner lumen. This is advantageous in order to provide the flexurally strong and/or kink-resistant properties of the catheter tubing over all the regions of the catheter tubing.

Furthermore, it is preferable in general if the catheter tubing is formed in one piece. This facilitates the production of the catheter tubing. In particular the catheter tubing may be formed as a one-piece injection-molded part. The outside diameter of the catheter tubing may vary along its length. In particular, the catheter tubing may also have a smaller outside diameter distally than proximally. In addition, the catheter tubing may be provided with the reinforcement, comprising a material different from the material of the catheter tubing.

At least one inner lumen of the catheter tubing preferably forms a through-duct. The through-duct can be used to insert medical instruments, for example, a thrombectomy device, a guide wire and/or a microcatheter, and advance them to the treatment site. It is especially advantageous if at least the through-duct has the reinforcement. In other words, the through-duct can be reinforced, so that it is prevented from collapsing and/or kinking when the catheter is guided through tight vascular curves in the vessels. This preserves good navigability for medical instruments.

In general, it is pointed out that, within the scope of the present invention, an insertion airlock is also considered to be a medical catheter, which is used to insert a catheter into the vascular system. In general, it is possible to provide that the through-lumen of the catheter can be used to supply smaller catheters accordingly, for example, microcatheters. In such a case, the outer catheter forms an insertion airlock.

A preferred embodiment of the invention provides that the inner lumen, in particular at least the through-duct, comprises an essentially circular basic cross-sectional shape. In determining the basic cross-sectional shape, any structuring that is present, for example, longitudinal grooves, is not taken into account in determining it. To design the basic cross-sectional shape of the individual inner lumen as a circle has the advantage that it allows medical instruments to be guided well to the site of treatment. Furthermore, the circular cross-sectional geometry permits rotation of medical instruments in the inner lumen, in particular in the through-duct. This may be advantageous, for example, to place the catheter by means of a navigable guide wire.

For use for hypothermia treatment, it is especially preferable if at least two inner lumens are formed as temperature control ducts, wherein the catheter tubing carries a heat exchanger element, in particular an expandable balloon. The heat exchanger element is preferably arranged on a distal catheter section and is thus fluidically connected to the temperature control ducts, so that a temperature control circulation is or can be formed.

On the whole, at least three inner lumens may be provided, wherein one inner lumen is designed as a through-duct and two other inner lumens are designed as temperature control ducts. The temperature control ducts may each form an inlet and a return, wherein the inlet and the return are fluidically connected through the heat exchanger element. A circulation through which a coolant flows may be provided by the temperature control ducts and the heat exchanger element, thereby achieving efficient cooling at the distal end of the catheter.

In a preferred embodiment of the invention, at least four inner lumens are provided. Of the four inner lumens, at least three inner lumens are preferably lined with a reinforcement. In concrete terms, all the inner lumens may be lined with a reinforcement.

In general, the reinforcements of neighboring inner lumens may be separated from one another by the material of the catheter tubing. The reinforcements of neighboring inner lumens are separated from one another to this extent.

A favorite embodiment of the catheter according to the invention comprises a catheter tubing having a through-duct and two temperature control ducts, wherein the through-duct has a multilayer reinforcement and the temperature control ducts each have a single-layer reinforcement. Therefore, in this embodiment, each one of the inner lumens has a reinforcement. The multilayer reinforcement is preferably constructed of three layers, wherein a middle layer consisting of a metal wire mesh and/or a polymer fiber mesh is embedded in an inner layer and an outer layer made of plastic. The temperature control ducts preferably have exclusively a single reinforcement, in particular a single-layer reinforcement.

In another embodiment, the reinforcement of an inner lumen, in particular the through-duct may protrude beyond the catheter tubing in some sections in a distal region of the catheter. In other words, the reinforcement of the at least one inner lumen in a distal region of the catheter, for example, is not surrounded by the catheter tubing and/or by a material of the catheter tubing. The at least one inner lumen may run distally independently of the other inner lumen and the catheter tubing. One or more reinforcements of at least one inner lumen may preferably extend over the total length of the inner lumen and/or outside of the catheter tubing in some sections. Therefore, the diameter of the catheter can be reduced in a distal region of the catheter. Within the scope of the present patent application, the distal region of the catheter having a reduced outside diameter is defined as the distal extension region.

At least one additional reinforcement is preferably provided for at least one inner lumen in a distal region of the catheter. Then at least one additional reinforcement in the distal region of the catheter may be associated with the one or more reinforcements extending over the entire length of the inner lumen or in sections outside of the catheter tubing. At least one additional reinforcement preferably has different mechanical properties than the catheter tubing. Then the flexibility of the catheter can be further increased in general.

Alternatively, it is conceivable for the catheter tubing to have a reduced number of inner lumens in some sections of a distal region of the catheter. Any combinations of the inner lumen and/or reinforcements are possible in general, so that the outside diameter of the catheter can be reduced in the distal region. The reinforcements may in general be formed by a plastic layer, in particular an inner or outer plastic layer or by a metallic reinforcement, in particular a metallic mesh or a metallic winding.

In a preferred embodiment, at least one reinforcement of one of the inner lumens in a proximal region of the catheter may protrude beyond the catheter tubing in some sections. In other words, the reinforcements of the inner lumen in a proximal region of the catheter are not surrounded by the catheter tubing and/or by a material of the catheter tubing. The inner lumens here may then run independently of one another. Therefore, the outside diameter of the catheter may also be reduced advantageously in a proximal region. In particular only a portion of the inner lumens, for example, the temperature control ducts, may have a reinforcement. Then the reinforcement may extend over the entire length of the respective inner lumen and/or outside of the catheter tubing in some sections. The inner lumens may each be advantageously connected to a Luer connection. Within the scope of the present patent application, the proximal region of the catheter having the protruding reinforcements is defined as the proximal extension region.

In a particularly preferred embodiment, at least one sensor, in particular a temperature sensor or a pressure sensor may be arranged in the distal region of the catheter. In addition, at least one marker element may be arranged in the distal region. The sensor and/or the marker element may be formed in the proximal and/or distal extension region of the catheter.

In addition, a reinforcement of the inner lumen, in particular of the centrally located through-duct, may be constructed of at least two materials in multiple layers. For example, the multilayer reinforcement may comprise an interior layer of plastic, i.e., directly adjacent to the inner lumen. The plastic is preferably formed from a friction-reducing material such as PTFE or other friction-reducing materials such as FEP or HDPE. The reinforcement may additionally comprise an exterior layer adjacent to the interior layer. The exterior layer may comprise a metal, for example. The exterior layer may be formed by a metal mesh or a metal winding, in particular a coil. Alternatively, the exterior layer may be made of a plastic. It is conceivable for the interior and/or exterior layer to be made of a friction-reducing material. The exterior layer of the multilayer reinforcement may be in direct contact with the catheter tubing.

In another embodiment, the multilayer reinforcement of the centrally located inner lumen may be formed from three layers. The exterior layer may comprise a plastic which is arranged adjacent to an interior layer of metal, for example. It is possible in particular for the innermost reinforcing layer to be a plastic layer, in particular a layer made of a plastic with a low friction property. A metal-reinforcing layer is preferably applied to this plastic layer. The outermost reinforcing layer may in turn be a plastic layer, in particular a plastic layer having a reduced friction property. If this multilayer reinforcement protrudes partially beyond the catheter tubing in the distal region and/or in the proximal region of the catheter tubing, then the multilayer reinforcement may have an additional outside reinforcement in these regions.

In addition, a reinforcement of the temperature control duct may preferably be formed in one layer. The one layer may then comprise a plastic. Alternatively, the reinforcement of the temperature control duct may be constructed in multiple layers. Then the reinforcement may be formed from two layers, with each layer comprising a plastic.

The catheter tubing preferably has at least two heat exchanger elements, in particular at least two expandable balloons. The catheter tubing may preferably also have at least three, in particular at least four heat exchanger elements. The maximum number of heat exchanger elements may advantageously amount to six, but any number of heat exchanger elements is also conceivable. The heat exchanger elements may be arranged in a serial pattern on the catheter tubing, for example. It is particularly advantageous if the heat exchanger elements are fluidically connected to the temperature control ducts, so that they can be exposed to the oncoming flow in series.

The region of the catheter tubing on which the heat exchanger elements are arranged may preferably have a smaller diameter than the region of the catheter tubing located proximally to the heat exchanger elements. The region of the catheter tubing on which the heat exchanger elements are arranged more preferably has a larger diameter than the region of the catheter tubing situated distally from the heat exchanger elements. In this case the diameter of the centrally located inner lumen and/or that of the through-duct may remain constant over the total length of the catheter. Thus, in the region of the catheter tubing where the heat exchanger elements are arranged, the diameter of the other inner lumens and/or of the temperature control ducts may be designed to be smaller than in the region of the catheter tubing proximal to the heat exchanger elements. The region of the catheter tubing where the heat exchanger elements are arranged may preferably be between 80 mm and 120 mm long, in particular between 90 mm and 110 mm long, in particular 100 mm long.

### BRIEF DESCRIPTION OF THE INVENTION

The invention is explained in greater detail below on the basis of exemplary embodiments with reference to the accompanying schematic drawings, in which:

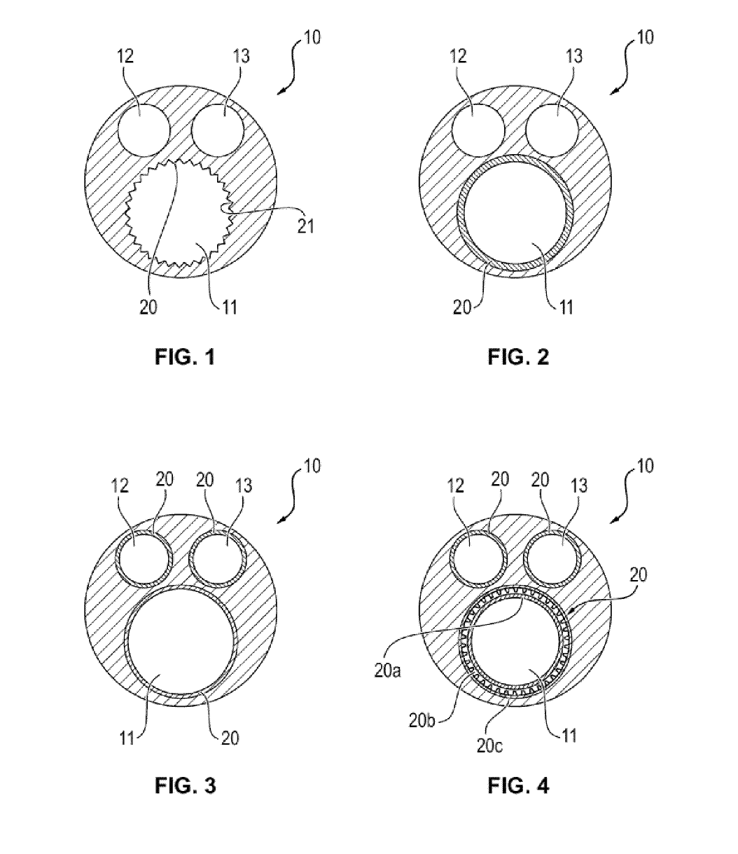

FIG. 1: shows how a cross-sectional view through the catheter tubing of a catheter according to the invention in a first exemplary embodiment;

FIG. 2: shows a cross-sectional view through the catheter tubing of a catheter according to the invention in a second exemplary embodiment;

FIG. 3: shows a cross-sectional view through the catheter tubing of a catheter according to the invention in a third exemplary embodiment; and

FIG. 4: shows a cross-sectional view through the catheter tubing of a catheter according to the invention in a fourth exemplary embodiment;

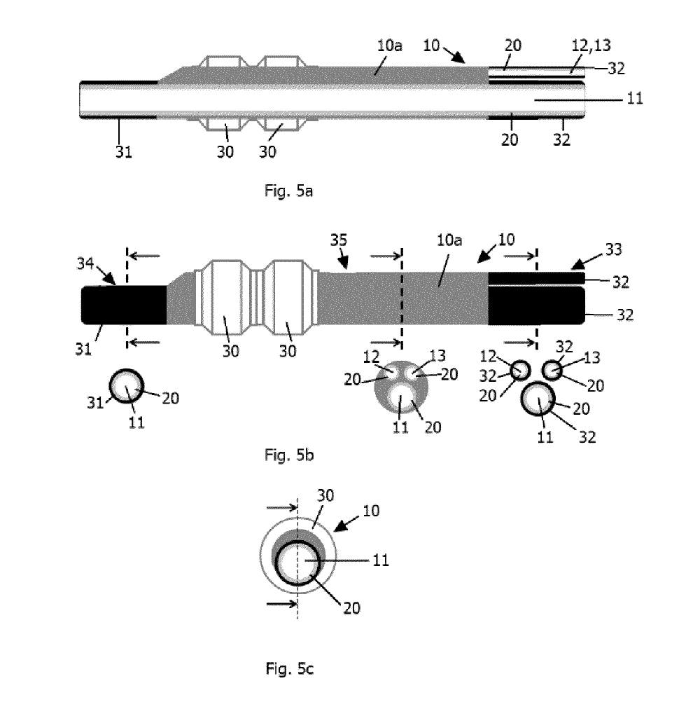

FIGS. 5a-c: show a longitudinal sectional view, a side view and a frontal view of a catheter according to the invention in a fifth exemplary embodiment.

### DETAILED DESCRIPTION OF THE INVENTION

The accompanying drawings each show a cross-sectional view through a catheter tubing 10, wherein the catheter tubing 10 has three inner lumens 11, 12, 13. Two inner lumens 12, 13 are designed as temperature control ducts 12, 13. The third inner lumen forms a through-duct 11.

All these exemplary embodiments have in common the fact that the through-duct 11 has a larger cross-sectional diameter than the temperature control ducts 12, 13. The inner lumens 11, 12, 13 of the through-duct 11 in particular and the temperature control ducts 12, 13 each have a circular cross-sectional basic shape. The catheter tubing 10 also has a circular cross-sectional outer contour.

The inner lumens 11, 12, 13 are separated from one another by the material of the catheter tubing 10. The catheter tubing 10 is preferably formed from a single material in one piece.

To increase the flexural stiffness of the catheter tubing 10, at least one of the inner lumens 11, 12, 13 is provided with a reinforcement 20. In the exemplary embodiment according to FIG. 1, the reinforcement 20 is formed by structuring.

The structuring is formed as a longitudinal grooved contour extending over the inside circumference of the through-duct 11. In cross section, the through-duct therefore has a gear-like cross sectional inner contour. The basic shape of the cross section of the through-duct 11 is still circular, however.

The longitudinal grooves 21 preferably extend over the total length of the through-duct 11. The distance between the individual longitudinal grooves in the circumferential direction of the inside circumference of the through-duct 11 is preferably regular.

FIG. 2 shows an alternative exemplary embodiment, wherein the through-lumen 11 is provided with a reinforcement 20, which is designed as a separate material layer. In concrete terms it is provided that an additional material, which acts as the reinforcement 20, is arranged over the inside circumference of the through-lumen 20. The additional material is different from the material of the catheter tubing 10. In particular the additional material of the reinforcement 20 is preferably flexurally strong. In particular the material of the reinforcement 20 may have a greater strength than the material of the catheter tubing 10.

The exemplary embodiment according to FIG. 3 differs from the embodiment according to FIG. 2 in that in addition to the through-duct 11, the temperature control ducts 12, 13 are also provided with a reinforcement 20. It is possible in general to provide for at least one of the inner lumens 11, 12, 13 to be furnished with a reinforcement 20. Two or more inner lumens 11, 12, 13 may also be provided with the reinforcement 20.

It is possible for the wall thickness of the reinforcement to be of a dimension such that the reinforcements 20 which are associated with different inner lumens 11, 12, 13 come in contact with one another. In other words, it is possible to provide that the individual inner lumens 11, 12, 13, in particular at least two inner lumens 11, 12, 13, are separated from one another by only the reinforcement 20. In this case, there is no separation by the material of the catheter tubing 10.

The exemplary embodiment according to FIG. 4 is based on the exemplary embodiment according to FIG. 3 and shows a catheter tubing 10 having three inner lumens 11, 12, 13, wherein inner lumens designed as temperature control ducts 12, 13 are provided with a reinforcement 20. A third inner lumen designed as a through-duct 11 also has a reinforcement 20.

The reinforcement 20 of the through-duct 11 differs from the reinforcements 20 arranged in the temperature control ducts 12, 13. In particular the reinforcement 20 of the through-duct 11 is designed in multiple layers.

Therefore, an inner layer 20a, which preferably comprises a friction-reducing material, is provided. In concrete terms, a plastic material can be used for the inner layer 20a. PTFE, FEP or HDPE is preferred.

An outer layer 20c, preferably formed by a thermoplastic polymer, is provided. The outer layer 20c is directly adjacent to the inside circumferential surface of the through-duct 11.

A middle layer 20b is arranged between the outer layer 20c and the inner layer 20a. The middle layer 20b is preferably formed by a metallic mesh structure, in particular a metallic woven mesh. Alternatively it is possible to provide for the middle layer 20b to comprise a metallic winding, for example, a coiling.

Instead of a metal, the middle layer may also comprise a plastic material. To this extent polymer fibers are preferred to form the winding or the mesh. In general the wires or fibers forming the mesh or the winding may be embodied as round wires and/or round fibers or as flat wires and/or flat fibers. In all three cases, the fibers may each be formed by multifilament wires.

The reinforcement 20 arranged in the temperature control ducts 12, 13 preferably comprises a polyimide material. In particular, polyimide tubings inserted into the temperature control ducts 12, 13 may be provided. The polyimide tubings are then preferably arranged in such a way that they are fixedly in contact with the inside circumference of the temperature control ducts 12, 13. As an alternative to the polyimide material, HDPE or PA may also be used as the material for the reinforcement 20.

For all embodiments of the medical catheter it is true that a thermoplastic material is preferably used as the material for the catheter tubing 10. For example, polyether block amide having a Shore hardness of at least 35 D, in particular at least 40 D, in particular at least 50 D, in particular at least 63 D, in particular at least 70 D, in particular at least 72 D may be used. The material of the catheter tubing 10 may be designed so that the Shore hardness increases from the distal catheter tip to the proximal shaft end of the catheter tubing. PA may also be used as the material for the catheter tubing 10 as an alternative to polyether block amide.

The temperature control ducts 12, 13 are preferably provided with a reinforcement made of PEEK, polyamide and/or nylon and/or polyimide. It is possible to provide that the reinforcement 20 of the temperature control ducts 12, 13 is designed in multiple layers. In particular the reinforcement 20 of the temperature control ducts 12, 13 may have additional stiffening due to a mesh and/or a wire winding.

The through-lumen 11 preferably has a reinforcement 20 formed by a thermoplastic material which is different from the material of the catheter tubing 10. The material of the reinforcement 20 can be differentiated from the material of the catheter tubing 10 in particular in the composition of the material and/or the Shore hardness. The reinforcement 20 of the through-duct 11 has a material thickness between 50 μm and 100 μm.

The reinforcement 20 may comprise additional stiffening by means of a mesh as illustrated in FIG. 4. The mesh preferably has round wires, wherein round wires having a cross-sectional diameter of at most 51 μm, in particular at most 40 μm, in particular at most 26 μm are used. When using flat wires, it is preferable for the flat wires to have a height of at most 51 μm and a width of at most 130 μm. Such wire dimensions may also be used for a winding, in particular a coil-type winding.

Preferred materials for the wires of the mesh and/or the winding include stainless steel or a nickel-titanium alloy.

The reinforcement 20, which is constructed in multiple layers, as illustrated in FIG. 4 as an example, preferably has a total thickness between 30 μm and 100 μm, in particular between 40 μm and 50 μm, in particular between 50 μm and 80 μm.

The through-lumen 11 is preferably of such dimensions that catheters and/or medical instruments in a size of 4 French, in particular 5 French, preferably 6 French, can be passed through it. In other words, the inside diameter of the through-lumen 11 having the reinforcement 20 is preferably at least 1.5 mm, in particular at least 1.8 mm, in particular at least 2.15 mm. In concrete terms, it is possible to provide for the through-lumen 11 to have an inside diameter in the range between 1.5 mm and 1.6 mm, in particular in the range from 1.8 mm to 1.9 mm, in particular in the range of 2.15 mm to 2.25 mm.

The temperature control ducts preferably have an inside diameter of at least 0.4 mm, in particular at least 0.6 mm, in particular at least 0.8 mm. The inside diameter of the temperature control ducts 12, 13 preferably amounts to at most 1.6 mm, in particular at most 1.4 mm, in particular at most 1.2 mm. It is particularly preferred if the temperature control ducts 12, 13 have an inside diameter of 1 mm.

The temperature control ducts 12, 13 preferably have a circular cross section. However, it is also possible for the temperature control ducts 12, 13 to have an oval, kidney-shaped or lung-shaped cross section.

The accompanying drawings show exemplary embodiments of the invention in which the catheter tubing 10 comprises three inner lumens 11, 12, 13. It is conceivable for more than three inner lumens 11, 12, 13 to be present. In particular at least one fourth inflation duct, which is connected to an inflatable balloon at the catheter tip, may be provided. The balloon is preferably designed as an occlusion balloon and makes it possible to close off a blood vessel. A fluid, for example, a gas or a liquid, can be transported through the inflation duct to the occlusion balloon, which results in expansion of the occlusion balloon.

Furthermore, in preferred exemplary embodiments of the catheter, it is possible to provide that a temperature sensor is arranged in the distal section of the catheter tubing 10, in particular at the catheter tip. The temperature sensor can be used to measure the blood temperature, so that the actual effect of the hypothermia can be monitored. Furthermore, a pressure sensor for the blood pressure measurement may be provided in a distal shaft section of the catheter tubing 10, in particular at the catheter tip. Other sensors, for example, a flow sensor are also conceivable.

With the arrangement of sensors in a distal shaft section of the catheter tubing 10, it is preferably provided that when the catheter tubing 10 has an additional lumen, it has in particular a cable duct for advancing electrical lines to the sensors.

Furthermore, a hydrophilic coating may be applied to the outside circumference of the catheter tubing 10. This improves the navigability of the catheter tubing 10 and/or of the catheter as a whole into a blood vessel. Furthermore, the hydrophilic coating, inasmuch as it is applied to a temperature control balloon, can improve the thermal efficacy of the hypothermia treatment. In particular, a heat exchange between the temperature control balloon and the surrounding blood is improved by the hydrophilic coating.

The material of the catheter tubing 10 can also be mixed with radiopaque and/or biocompatible powder. Such a powder can be introduced into the material of the catheter tubing 10 in particular. For example, approx. 10 wt % to 20 wt % of the material for the catheter tubing 10 may be formed by such a radiopaque and/or biocompatible powder. This improves the radiopacity of the catheter tubing 10 in particular. For this purpose, materials such as barium sulfate, bismuth trioxide, bismuth carbonate, zirconium, tungsten and/or tantalum are suitable in particular.

FIGS. 5a-c each show a longitudinal sectional view, a side view and a frontal view of a catheter according to the invention in another exemplary embodiment. The catheter tubing 10 has three inner lumens 11, 12, 13. Two inner lumens 12, 13 are designed as temperature control ducts 12, 13. The third inner lumen forms a through-duct 11. The inner lumens 11, 12, 13 are separated from one another by the material 10a of the catheter tubing. The material 10a of the catheter tubing may preferably have a thermoplastic elastomer such as PEBA or polyurethane.

Each of the inner lumens 11, 12, 13 has a reinforcement 20. The reinforcement 20 may be designed as a separate material layer. The additional material may be different from the material 10a of the catheter tubing. For example, the reinforcement 20 may be formed of a plastic. For example, the reinforcement 20 has a friction-reducing material. The reinforcement 20 may preferably be formed from a plurality of layers, in particular three layers. The interior layer and/or the layer adjacent to the inner lumen may comprise a plastic of a friction-reducing material, in particular fluoropolymer, PTFE or FEB. The layer in the middle may preferably be formed from a material reinforcement. For example, the material reinforcement may be braided or may have a coil. The exterior layer may preferably comprise a thermoplastic elastomer such as PEBA or polyurethane. Thus the exterior layer of the reinforcement 20 may also comprise the same material as the material of the catheter tubing 10a.

The catheter has a proximal extension region 33 and a distal extension region 34. The distal extension region 34 may have a length of 10 mm to 100 mm, in particular of 20 mm to 80 mm, in particular of 30 mm to 70 mm, preferably of 50 mm. The outside diameter of the catheter may amount to 3 mm in particular in the distal extension region 34. Thus the catheter may have an outside diameter of 9 French in particular in the distal extension region 34. The proximal extension region 33 may have a length of 10 mm to 100 mm, in particular of 20 mm to 80 mm, in particular of 30 mm to 70 mm, preferably of 50 mm.

In addition, the catheter has a middle region 35, arranged between the distal extension region 34 and the proximal extension region 33. The outside diameter of the catheter in the middle region 35 may be 2 mm to 5 mm, in particular 2.5 mm to 4.0 mm, in particular 3.5 mm. Thus the catheter may have an outside diameter of 11 French, in particular in the middle region 35. The entire shaft region of the catheter may preferably be of such dimensions that catheters and/or medical instruments with a size of 4 French, in particular 5 French, preferably 6 French, can be passed through it. The catheter may in general have a total length of 70 cm to 120 cm, in particular of 80 cm to 110 cm, preferably of 90 cm.

In the distal extension region 34, the reinforcement 20 of the through-duct 11 protrudes in sections beyond the catheter tubing 10. In other words, the reinforcement 20 of the through-duct 11 in the distal extension region 34 of the catheter, for example, is not surrounded by the material 10a of the catheter tubing. The outside diameter of the catheter tubing 10 is then reduced in the distal extension region 34. The reinforcement 20 forms an extension of the catheter tubing 10 in the distal extension region 34.

In addition, an additional distal reinforcement 31 is arranged in the distal extension region 34. Then the distal reinforcement 31, as can be seen in FIG. 5a, is arranged as an additional layer on the reinforcement 20 of the through-duct 11. For example, the reinforcement 20 of the through-duct 11 may be formed as a plastic layer. The distal reinforcement 31 may also comprise, for example, a plastic, in particular a plastic design to be flexible. For example, the reinforcement 31 may comprise a plastic having a lower modulus of elasticity and/or a lower hardness than the material of the catheter tubing 10a. The reinforcement 31 may preferably comprise a thermoplastic elastomer, such as PEBA or polyurethane.

In the proximal extension region 33, the reinforcements 20 of the inner lumens 11, 12, 13 protrude beyond the catheter tubing in some sections. In other words, the reinforcements 20 of the inner lumens 11, 12, 13 are not surrounded by the material of the catheter tubing 10a in the proximal extension region 33 of the catheter. The inner lumens 11, 12, 13 run independently of one another. Thus the outside diameter of the catheter may advantageously also be reduced in a proximal region. Furthermore, a facilitated connecting, for example, of Luer connections to be attached may be achieved due to the independence of the inner lumens 11, 12, 13 of one another.

For example, two balloons 30 are arranged on the outside circumference of the catheter tubing 10. The balloons 30 are arranged serially on the catheter tubing 10. It is particular advantageous if the balloons 30 are fluidically connected to the temperature control ducts 12, 13 such that they can be exposed serially to the oncoming flow.

### LIST OF REFERENCE NUMERALS

10 catheter tubing
10a material of the catheter tubing
11 through-duct
12, 13 temperature control duct
20 reinforcement
20a inner layer
20b middle layer
20c outer layer
30 balloon
31 distal reinforcement
32 proximal reinforcement
33 proximal extension region
34 distal extension region
35 central region

1. A medical catheter for hypothermia treatment with a catheter tubing, the medical catheter comprising:
a first inner lumen and a second inner lumen, the first inner lumen forming a temperature control duct adapted for conveying a cooling liquid, and
a reinforcement associated with the first inner lumen or the second inner lumen.

2. The catheter according to claim 1,
wherein the second inner lumen forms a through-duct, the through-duct being separated from the temperature control duct, and
wherein the reinforcement is associated with the through-duct or the temperature control duct.

3. The catheter according to claim 1, wherein the reinforcement lines the first inner lumen or the second inner lumen and comprises a material different from a material of the catheter tubing.

4. The catheter according to claim 1, wherein the reinforcement comprises a structuring or a grooved longitudinal contour.

5. The catheter according to claim 4,
further comprising the catheter tubing,
wherein
the structuring is formed in the material of the catheter tubing or in the material of the reinforcement.

6. The catheter according to claim 1,
wherein the reinforcement comprises two materials in multiple layers or more than two materials in multiple layers.

7. The catheter according to claim 1, wherein
the reinforcement has a metallic mesh or a metallic coil.

8. The catheter according to claim 1, wherein
the reinforcement extends over an entire inside circumference of the first inner lumen or an entire inside circumference of the second inner lumen.

9. The catheter according to claim 1, wherein
the reinforcement extends over an entire length of the first inner lumen or an entire length of the second lumen.

10. The catheter according to claim 1,
further comprising the catheter tubing,
wherein
the catheter tubing is formed in one piece.

11. The catheter according to claim 1, wherein
at least one inner lumen forms a through-duct.

12. The catheter according to claim 1,
the through-duct has an essentially circular cross-sectional basic shape.

13. The catheter according to claim 1,
further comprising the catheter tubing;
wherein
the first inner lumen and the second inner lumen are temperature control ducts wherein the catheter tubing carries a heat exchanger element that is arranged on a distal catheter section of the catheter tubing and is fluidically connected to the temperature control ducts for a temperature control circulation
wherein the heat exchanger element is an expandable balloon.

14. The catheter according to claim 1,
further comprising a third inner lumen and a fourth inner lumen,
wherein at least three inner lumens are lined with the reinforcement.

15. The catheter according to claim 14,
further comprising the catheter tubing;
wherein
the reinforcements of neighboring inner lumens are separated from one another by the material of the catheter tubing.
